(19) United States
(12) Patent Application Publication
(10) Pub. No.: US 2021/0386119 A1
(43) Pub. Date: Dec. 16, 2021

(54) An Aerosol Generating Device

(71) Applicant: JT International S.A., Geneva (CH)

(72) Inventors: Marko Plevnik, London (GB); Norihiko Inoue, London (GB); Jamie Buckley, Ditton (GB); Layth Sliman Bouchuiguir, Bellevue (CH)

(73) Assignee: JT International S.A., Geneva (CH)

(21) Appl. No.: 17/291,830

(22) PCT Filed: Nov. 19, 2019
(86) PCT No.: PCT/EP2019/081858

§ 371 Date: May 6, 2021

(30) Foreign Application Priority Data
Nov. 19, 2018 (EP) 18207091.2

(51) Int. Cl. A24F 40/46; A24F 40/60; A24F 40/42; A24F 40/30

(52) U.S. Cl. CPC A24F 40/46; A24F 40/30; A24F 40/42; A24F 40/60

(57) ABSTRACT

An aerosol generating device includes a heating chamber arranged to receive a consumable and heat the consumable to generate an inhalable vapour; a consumable picker arranged to engage and retain a consumable, wherein the consumable picker is movable relative to the heating chamber between: a first, external position in which the consumable picker is positioned external to the device; and a second, internal position in which the consumable picker lies within the heating chamber; such that a user may engage a consumable with the consumable picker in the first position and subsequently move the consumable picker to the second position, thereby moving the consumable picker with the retained consumable into the heating chamber for heating. By providing an aerosol generating device including a consumable picker configured in this way, the process of exchanging the consumable is greatly simplified.

### TECHNICAL FIELD

The present disclosure relates to an aerosol generating device and an aerosol generating system comprising an aerosol generating device and consumable.

### TECHNICAL BACKGROUND

Aerosol generating devices such as electronic cigarettes are relatively well known and are becoming increasingly popular with consumers in recent years. A common operating principle for such electronic cigarettes is to heat a consumable without burning it to provide an aerosol (also referred to as a vapour) to a user for inhalation. Examples of such consumables include charges of tobacco material or capsules of liquid containing flavourants and active ingredients such as nicotine.

Aerosol generating devices often comprise a heater, a power source for supplying electricity to the heater and a receptacle, such as a heating chamber, for receiving the consumable in the vicinity of the heater such that the consumable may be heated to produce the vapour for inhalation.

In recent years the popularity of multiple use aerosol generating devices has surpassed that of single use devices which are disposed of after use. Since, the consumable can only provide the required active ingredients or flavourants for a limited duration of heating before these components are depleted, in multiple use devices the consumable must be replaced. This is generally achieved by removing the depleted consumable from the heating chamber and replacing with a new consumable.

There are however several shortcomings with such aerosol generating devices and systems. In particular, the replacement of the consumable is often an awkward process for the user, given the size of the components and the requirement to manoeuvre the aerosol generating device into the appropriate orientation while removing the spent consumable and replacing with a new consumable. This process has to be carried out regularly which exacerbates the problem. Furthermore, the process generally requires the consumable to be removed by hand, where the user has to directly touch the consumable. This can lead to the materials of the consumable being left on the skin or staining clothing which is clearly undesirable. In the case of consumables formed of charges of compressed smokeable material, after use the depleted material can fall apart easily meaning it can be difficult to handle and is liable to fall apart when touched.

Therefore, there is need to provide an aerosol generating system which addresses one or more of these shortcomings.

### SUMMARY OF THE DISCLOSURE

According to a first aspect of the present disclosure, there is provided an aerosol generating device comprising: a heating chamber arranged to receive a consumable and heat the consumable to generate an inhalable vapour; a consumable picker arranged to engage and retain a consumable, wherein the consumable picker is movable relative to the heating chamber between: a first, external position in which the consumable picker is positioned external to the device; and a second, internal position in which the consumable picker lies within the heating chamber; such that a user may engage a consumable with the consumable picker in the first position and subsequently move the consumable picker to the second position, thereby moving the consumable picker with the retained consumable into the heating chamber for heating.

By providing an aerosol generating device including a consumable picker configured in this way, the process of exchanging the consumable is greatly simplified. In particular, since a component of the device itself may be used to pick a consumable and insert it into the heating chamber, the amount of dexterity required to perform the exchange is reduced, since the user does not have to handle the relatively small consumable and orientate it correctly by hand. Furthermore, the fact that the consumable remains engaged by the consumable picker when in the heating chamber means that a further step of removing the consumable picker from the consumable is not required. Advantageously, the full consumable exchange process may be performed without any direct user contact with the consumable such that the risk of contamination of the skin or staining of clothing is reduced.

The term “consumable picker” is intended to refer to a component of the aerosol generating device which is configured such that it may pick up a consumable (such as a charge of compressed aerosol generating substance, or a cartridge containing an aerosol generating substance) and move the consumable into the heating chamber. In particular the consumable picker is configured to directly pick up a consumable and move the consumable into the chamber without any additional user input. In other words, the consumable picker is configured to engage a consumable and retain it sufficiently such that the consumable may be directly picked up and moved into the chamber. The consumable picker is configured to sufficiently retain the consumable such that, once engaged, the consumable may be lifted and moved to the heating chamber. It can take any appropriate form, depending on the specific form of the consumable, to provide this function. For example it may comprise means to clasp, envelop, pierce or otherwise engage with the consumable in order to retain the consumable sufficiently to allow for movement between the first and second positions while retaining the consumable. For example it may be a claw, an array of prongs which surround the consumable, a receptacle for receiving the consumable or one or more protrusions for spearing the consumable or to be received within a corresponding hole or holes in the consumable.

The “first, external position” refers to a position or state (which might include multiple positions) in which the consumable picker is positioned external to the device in a manner that allows a consumable to be engaged. For example, when the consumable picker forms part of a removable heating chamber lid, the first position is any position in which the lid is held, when it has been removed from the device by the user, such that the user may engage the consumable and return the lid to the heating chamber thereby moving the consumable to the second position. “Moveable” thus encompasses being movable by a user and being movable by a component of the device.

The “second, internal position” refers to a position in which the consumable picker is retained within the heating chamber. In particular, in the second position the consumable picker is supported by the device such that it lies within the heating chamber.

Preferably the consumable picker comprises a protrusion. The protrusion may be arranged to spear the consumable in order to retain in or be arranged to fit into a corresponding opening or hole in the consumable. This provides a straightforward means for a user to reliably retain the consumable with the consumable picker which requires little dexterity.

In certain examples of the invention the protrusion has a tapered shape. In particular, it may have a broader base and taper towards a thinner tip. In this way, as the protrusion is engaged with the consumable by a user and moved progressively deeper into the consumable, the increasing cross-section of the protrusion provides an increased frictional retaining force, either with the internal material of the consumable as it spears it or with the internal surfaces of an opening or through hole in the consumable. Furthermore a tapered protrusion allows for the user to discard a spent consumable with ease since the thinner end allows the consumable to be dislodged with minimal force.

In order to further improve the retaining capability of the consumable picker, the protrusion may also have a textured surface, friction coating or user activated engagement means (such as one or more additional extending protrusions) which engage the consumable.

In certain example of the invention, the consumable picker is a protrusion which is provided on a piston moveable between the first and second position. In particular, the piston may move within the heating chamber such that the protrusion is fully retracted within the heating chamber in the second position and extends out of the heating chamber, external to the device, in the first position. The device may have an elongate axis with the piston moving within the elongate body in a direction corresponding to the elongate axis of the device. Movement of the piston may be controllable with a user actuation means.

In certain examples of the invention, the aerosol generating device further comprises a heating chamber lid, moveable between an open position to allow access to the heating chamber and closed position in which the heating chamber is enclosed; wherein the consumable picker is coupled to the heating chamber lid such that movement of the lid between the open and closed position moves the consumable picker between the external and internal position to simultaneously insert the consumable picker into the chamber and close the heating chamber opening. In this way, a compact arrangement is provided in which the number of required parts is reduced and the exchange process is simplified. In particular, movement of the consumable picker into the heating chamber simultaneously closes the heater chamber ready for heating, so that no additional step of closing the heating chamber is required.

In such examples of the invention, preferably the consumable picker forms part of the heating chamber lid and extends from an internal surface of the heating chamber lid such that when the heating chamber lid is in the closed position the consumable picker extends into the heating chamber. In such an arrangement the number of required parts is reduced since the lid of the heating chamber also functions as the consumable picker. This also reduces the dexterity required to perform the exchange process as the user need only hold the aerosol generating device and the lid and so does not require more than two items to be held and manipulated simultaneously.

The lid comprising the consumable picker may be a detachable part of the device which the user can move freely to engage the consumable before returning the lid to the device. Because the lid is easier to handle and to correctly orientate in comparison to the device as a whole, the consumable exchange process is made more user friendly as only the lid needs to be orientated to engage the consumable.

Alternatively, the consumable picker is a separate component to the lid and the consumable picker and the lid are mechanically coupled such that movement of the consumable picker between the external and internal position drives movement of the lid between the open and closed position and/or movement of the lid between the open and closed position drives movement of the consumable picker between the external and internal position. In this way, the lid remains attached to the device such that it cannot be lost. Furthermore, the device can be used one handed as there is no requirement to handle the lid. Preferably the device comprises a user actuation means arranged to drive the movement of the heating chamber lid and the consumable picker. In this way, the user can use one hand to hold the device and activate the user actuation means to retract the lid and extend the protrusion after which the consumable may be engaged and the actuation means used again to return the picker and consumable to the chamber. Thus the process may be performed with one hand and is greatly simplified. The mechanical coupling between the lid, consumable picker (and, if included, the actuation means) may be provided by an array of gears which couple the movement of the lid to movement of the consumable picker between the first and second position.

In certain examples of the invention where the consumable picker is provided as part of a piston arranged to move between the first and second position, the piston is mechanically coupled to the lid such that movement of the lid from the open to the closed position simultaneously moves the consumable picker from the first to the second position. This coupled movement may be driven by a user actuation means.

In certain example of the present invention the device further includes an elongate body and a sleeve, the elongate body moveable within the sleeve in a direction corresponding to the elongate axis; wherein the consumable picker forms part of the body with the protrusion extending from one end of the body; such that relative movement of the sleeve and body provides movement between the first position in which the protrusion extends out of the sleeve and the second position in which the protrusion is retracted within the sleeve. This provides a compact arrangement in which the movement between the first and second position may be achieved with one hand by movement of the sleeve relative to the “core” body component within. Furthermore the protrusion may be directly connected to the body providing a sturdy connection which is less liable to break. Attaching the protrusion directly to the body also allows for ease of connection to the battery such that it may function as a heater as described below. In these examples the heating chamber may be provided by the enclosed volume defined by the end of the body at which the protrusion is connected and the internal surfaces of a sleeve. The device may also include a lid for enclosing the chamber. Alternatively the consumable may be provided with a mouthpiece such that when the consumable is engaged with the protrusion and retracted within the chamber formed by the sleeve, a portion of the consumable extends through an end opening of the sleeve to provide a mouthpiece through which the vapour may be inhaled.

In certain examples of the invention the consumable picker comprises a heating element. This reduces the complexity of the device in terms of a reduction in the number of components required. Furthermore, in the case that the consumable picker comprises a protrusion which enters the consumable, it allows for efficient heating of the consumable as the the consumable is heated from within. This provides more uniform heating of the consumable than if the consumable is heated externally. Furthermore heating the consumable from within is more efficient as less heat escapes and accordingly the heating chamber does not need to be as well insulated, reducing costs.

In certain examples the device further comprises a releasing means configured to release the consumable from consumable picker upon actuation by the user. In this way, the user may simply release the consumable from the picker when it is spent, simplifying the exchange process further. The release means may be a mechanism by which the protrusion or part of the protrusion can be retracted such that it is withdrawn from the consumable. Alternatively the release means is provided by a consumable engagement element arranged to engage the consumable when it is retained by the consumable picker and remove the consumable from the consumable picker. For example, the consumable engagement element may be provided on the inner facing side of the heat chamber lid such that, when the consumable is retained on the protrusion within the chamber and the lid is closed, the engagement element enters the chamber to grip the consumable. Subsequent movement of the lid to the open position then extracts the consumable from the consumable picker. For another example, the consumable engagement element may be provided on the inner facing side of the heat chamber lid such that, when the consumable is retained on the protrusion within the chamber and during the movement of the lid from closed position to open position, the engagement element catches the consumable and then extracts the consumable from the consumable picker. The engagement element is preferably a grip/catch arranged to grip/catch the outer sides of consumable so as not to contact the consumable picker when it is in the form of a protrusion within the consumable.

In another aspect of the invention there is provided an aerosol generating system comprising an aerosol generating device as described above and a consumable formed such that it will be retained after being engaged by the consumable picker. The aerosol generating device has the essential features described in claim 1 and optionally any combination of the features described above. The specific form of the consumable will be configured to that of the consumable picker used. For example it may have appropriate properties such that it can be speared by a protrusion and retained thereon. Alternatively it may simply be sized appropriately such that protrusions of the picker may fit around it in order to retain the consumable between the protrusions. Alternatively it may have a metallic component corresponding to a magnetic component of the protrusion such that it may be retained by magnetic force.

The aerosol generating device may be configured for use with various types of consumables containing an aerosol generating substance. The consumable may be a capsule which includes in use an aerosol generating substance inside an air permeable material. Alternatively, the consumable may be an aerosol generating substance held inside a material that is not air permeable, but which comprises appropriate perforation or openings to allow air flow.

Alternatively, the consumable may be the aerosol generating substance itself. Alternatively, the body of the consumable is formed substantially in the shape of a stick which may have a mouthpiece filter. In this case the consumable may be a sheet such as paper wrapped the aerosol generating substance.

The material including the aerosol generating material may have a high air permeability to allow air to flow through the material with a resistance to high temperatures. Examples of suitable air permeable materials include cellulose fibres, paper, cotton and silk. The air permeable material may also act as a filter. Alternatively, the body may be the aerosol generating substance wrapped in paper.

The aerosol generating substance may be any suitable substance capable of forming an aerosol. The substance may be solid or semi-solid substance. The substance may comprise plant derived material and in particular, the substance may comprise tobacco. Typically, the aerosol generating substance is a solid or semi-solid tobacco substance. Example types of aerosol generating solids include powder, granules, pellets, shreds, strands, porous material, foam or sheets.

Preferably, the aerosol generating substance may comprise an aerosol-former. Examples of aerosol-formers include polyhyrdric alcohols and mixtures thereof such as glycerine or propylene glycol. Typically, the aerosol generating substance may comprise an aerosol-former content of between approximately 5% and approximately 50% on a dry weight basis. Preferably, the aerosol generating substance may comprise an aerosol-former content of approximately 15% on a dry weight basis.

Preferably the consumable picker comprises a protrusion and the consumable has a central cavity shaped such that the protrusion of the consumable picker may be inserted into the cavity and the consumable retained on the protrusion. The cavity may be a through hole for example the consumable may be substantially cylindrical or ring shaped. This provides a straightforward means to allow the consumable to be releasably retained which is easy to manufacture.

In some examples the aerosol generating system further comprises a consumable package arranged to hold a plurality of consumables, each in a corresponding orientation such that a face of the consumable which is capable of being engaged with the consumable picker faces outwards, allowing a user to select a consumable by engaging the face with the protrusion of the consumable picker. In this way the exchange process is further simplified since the cavities of the protrusion are in an arrangement in which they are presented to the user for easy engagement with the protrusion. In some examples the consumables each have a cavity and the consumable picker comprises a protrusion, wherein the consumable package is arranged to hold the consumables such that the cavity faces outwards to allow the cavity to be engaged with the protrusion and the consumable lifted out of the package.

In some examples the consumable comprises a mouthpiece such that, when the consumable is engaged by the consumable picker and moved to the internal position within the chamber, the mouthpiece extends outside of the chamber allowing a user to inhale the generated vapour through the consumable. In this way the number of components are reduced since an additional mouthpiece or lid to chamber are not required.

### BRIEF DESCRIPTION OF THE DRAWINGS

FIG. 1 is a schematic illustration of an aerosol generating system according to the present invention;

FIG. 2 is a schematic illustration of the operation of a consumable picker of the aerosol generating device according to the present invention;

FIG. 3 shows further views of an aerosol generating device according to the present invention;

FIG. 4 shows a consumable package of the aerosol generating system according to the present invention;

FIG. 5 illustrates the consumable exchange procedure using an aerosol generating system according to the present invention;

FIG. 6A is a schematic illustration of another example of an aerosol generating system according to the present invention;

FIG. 6B is a schematic illustration of another example of an aerosol generating system according to the present invention;

FIG. 7 is a schematic illustration of another example of an aerosol generating system according to the present invention;

FIG. 8 is a schematic illustration of another example of an aerosol generating system according to the present invention.

### DETAILED DESCRIPTION OF EMBODIMENTS

Embodiments of the present disclosure will now be described by way of example only and with reference to the accompanying drawings.

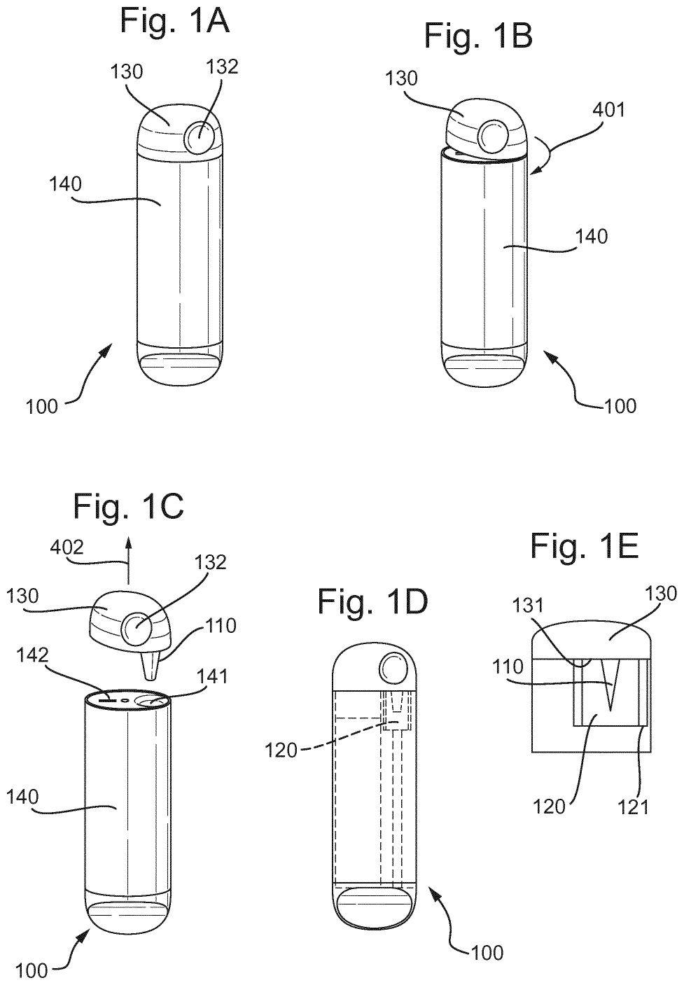

FIG. 1A-E schematically illustrates an aerosol generating device 100 according to the present invention. The device 100 includes a heating chamber 120 arranged to receive a consumable 200 and heat the consumable 200 to generate an inhalable vapour. The heating chamber 120 is shown in the internal view of FIG. 1D and the magnified schematic illustration of FIG. 1E. The device 100 further includes a consumable picker 110 arranged to engage and retain a consumable 200, wherein the consumable picker 110 is moveable relative to the heating chamber 120. In particular the consumable picker 110 is moveable between a first external position in which the consumable picker 110 is positioned external to the device 100, as shown in FIG. 1C, and a second, internal position in which the consumable picker 110 lies within the heating chamber 120, as most clearly shown in FIG. 1E. In this way, a user may engage a consumable 200 with the consumable picker 110, when it is external to the device 100 in the first position, as shown in FIG. 1C. The user then may subsequently move the consumable picker 110 to the second, internal position, thereby moving the consumable picker 110 with the retained consumable 200 into the heating chamber 120 for heating.

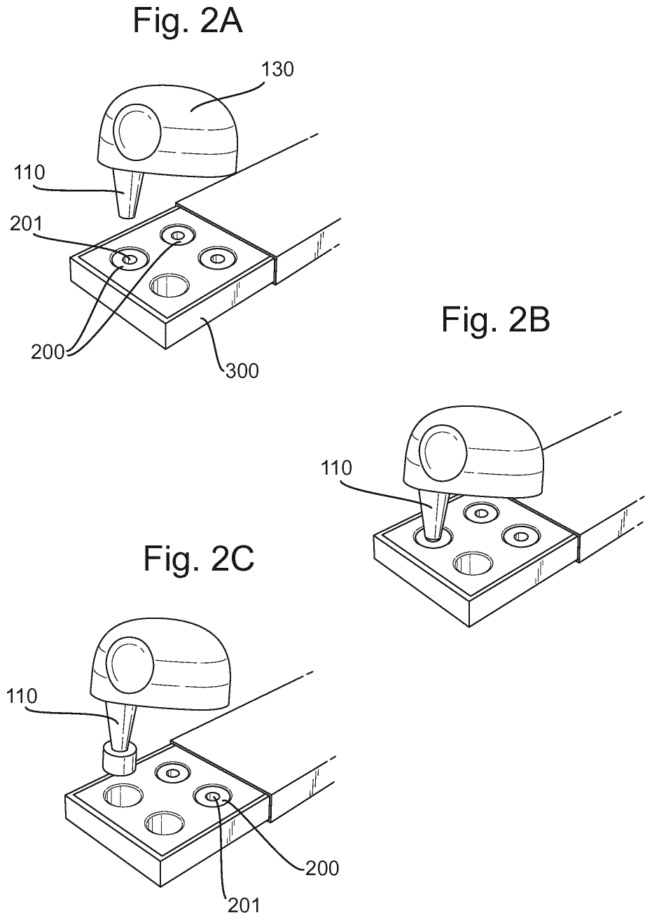

As shown in FIG. 1C and 1E in this example of the invention the consumable picker comprises a protrusion 110 which is configured to engage so as to releasably retain a consumable 200. The process of engaging a consumable 200 is illustrated in FIG. 2A-C. In particular, the protrusion is arranged such that it is appropriately sized to be inserted into a cavity 201 in the consumable 200 in order to releasably retain the consumable 200 on the protrusion 110 as shown in FIG. 2C. The protrusion 110 has a tapered shape such that, by engaging the protrusion 110 with the cavity 201 of the consumable 200 and applying a force to move the protrusion into the opening 201 the tapered shape of the protrusion 110 means that as the protrusion enters the opening the increasing cross section results in the frictional force between the protrusion and consumable 200 increasing in order to reliably retain the consumable 200 on the protrusion 110. This process is illustrated in FIGS. 2A-C in which the protrusion 110 is initially held above the consumable 200, as shown in FIG. 2A, the protrusion 110 is then brought progressively into the cavity 201 of the consumable 200 as shown in FIG. 2B, and finally the protrusion 110 may then be lifted away, with the frictional engagement force holding the consumable 200 on the protrusion 110, as shown in FIG. 2C.

In other examples the consumable may not have any specific shape and the protrusion may engage the consumable by simply penetrating the constituent material of the consumable in order to retain it on the protrusion.

In the example of FIGS. 1-3 the device 100 also includes a heating chamber lid 130 and the protrusion 110 extends from an internal surface of the lid such that when the lid is moved to the closed position, enclosing the heating chamber 120, the protrusion 110 lies within the heating chamber 120 as shown in FIG. 1E. In this example, the lid 130 is a detachable component, as shown in FIG. 1.

In particular, in this example, the lid may be removed from a fully closed position as shown in FIG. 1A by first twisting the lid 130 relative to the body 140 of the device 100 as shown by arrow 401 to unlock the lid 130. The lid 130 may then be raised in direction 402 to remove the lid from the body 140. The first external position therefore corresponds to any position in which the lid 130 is removed from the body 140 as shown in FIG. 1C, such that a consumable may be engaged. The “second position” is the position in which the heating chamber lid is returned to close the opening to the heating chamber, as shown in FIG. 1A.

The device 100 has an end surface 142 with a heating chamber opening 141. Once a consumable 200 has been selected with the protrusion 110, using the lid 130 to orientate the protrusion 110, the lid is returned to the device such that the protrusion 110 with the retained consumable 200, as shown in FIG. 2C enters the opening 141 and, as the protrusion 110 extends from an inner surface 131 of the lid. The protrusion 110 and the consumable 200 are thereby enclosed within the heating chamber 120 as shown in FIG. 1E. The lid 130 may have a grip or flattened finger hold 132 to facilitate these movements and orientation of the lid 130 (and accordingly the protrusion 110).

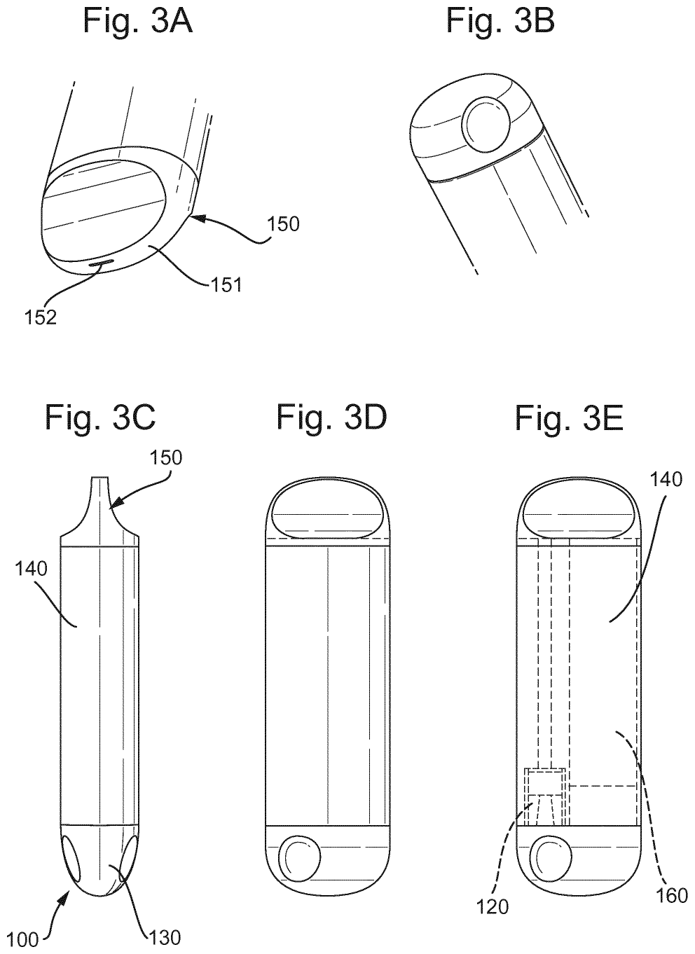

The further features of this example of the invention will now be described with reference to FIGS. 1 and 3. As shown in FIG. 3A-E, the aerosol generating device 100 has an elongate body 140 with the heating chamber lid 130 at one end of the body 140 and a mouthpiece 150 at the other end. As shown in FIG. 3A, the mouthpiece 150 has a tapered shape extending into a flattened protruding mouthpiece portion 151 around which the user can place their lip to inhale through outlet 152. The internal view of the device in FIG. 3E shows that, within the body 140, there is a battery 160 connected to the heaters 121 of the heating chamber 120. There is also a vapour passage extending between the heating chamber and the outlet 152 of the mouthpiece 150. In this example, the heaters 121 are provided around the heating chamber 120 as shown in FIG. 1E. The device 100 may further include an activation means such as a switch or button (not shown) to allow the user to selectively apply power from the battery to the heater in order to heat the consumable 200 to produce the vapour, which is then inhaled through the outlet 152 of the mouthpiece 150.

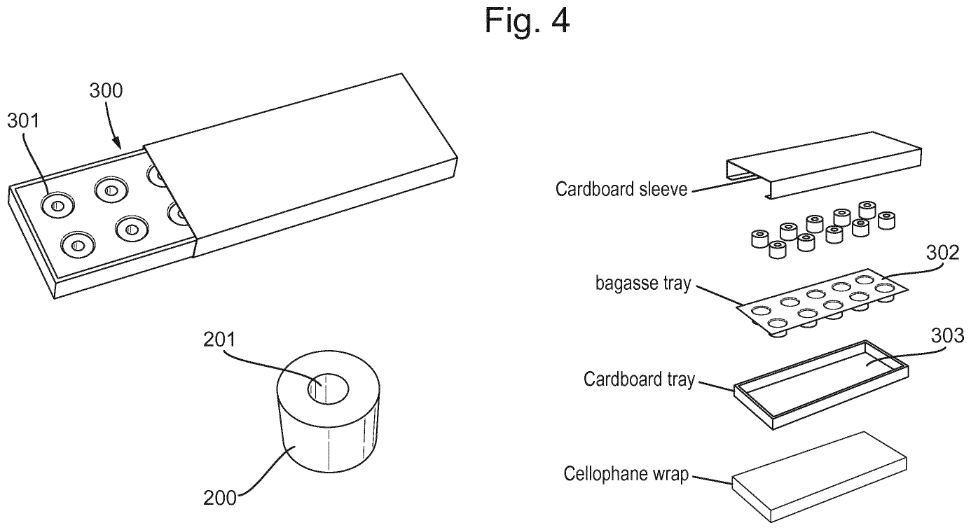

FIG. 4 shows a consumable package 300 arranged to hold a plurality of consumables 200 which may form an aerosol generating system together with the aerosol generating device FIG. 1. FIG. 4 shows the specific form of one possible consumable 200 for use with the above described aerosol generating device. As shown in the enlarged view of the consumable 200, it has a substantially cylindrical shape within an axial through hole 201 of appropriate sized to be releasably retained when engaged with the protrusion 110 of the current invention. It would be appreciated that the consumable could equally not comprise an opening or through hole but instead the protrusion of the picker could be arranged to spear the constituent material of the consumable in order to retain it.

The consumable may be formed of any suitable aerosol generating substance, as is known in the field. For example it may be a compressed charge of particles of smokeable material within a substance used to allow the charge to hold its form. In this example the consumable is a foam tobacco material.

The consumable package 300, shown in FIG. 4, has an array of recesses 301 of appropriate size to hold a consumable 200. In this example, the recesses 301 are sized such that they conform to the cross sectional shape of the cylindrical consumable 200, such that the axial cavities 201 are all aligned and arranged such that the face outwards, as shown in FIG. 4. This may be achieved with a tray 302 having an array of recessed cup-shaped positions in which the consumables 200 may be placed. This tray may be held within a conventional tray or box as shown in FIG. 4. By arranging the consumables in this way they may be easily accessed and engage with the consumable picker 110, without having to move or re-orientate the consumables 200.

The tray 302 may preferably be made from bagasse. Bagasse is derived from sugarcane and is a by-product of sugar manufacture. It is used for making pulp packaging, plates and drinks vessels. Unlike other kinds of paper, bagasse kitchen products don't need a plastic lining to stay waterproof and have good barrier properties.

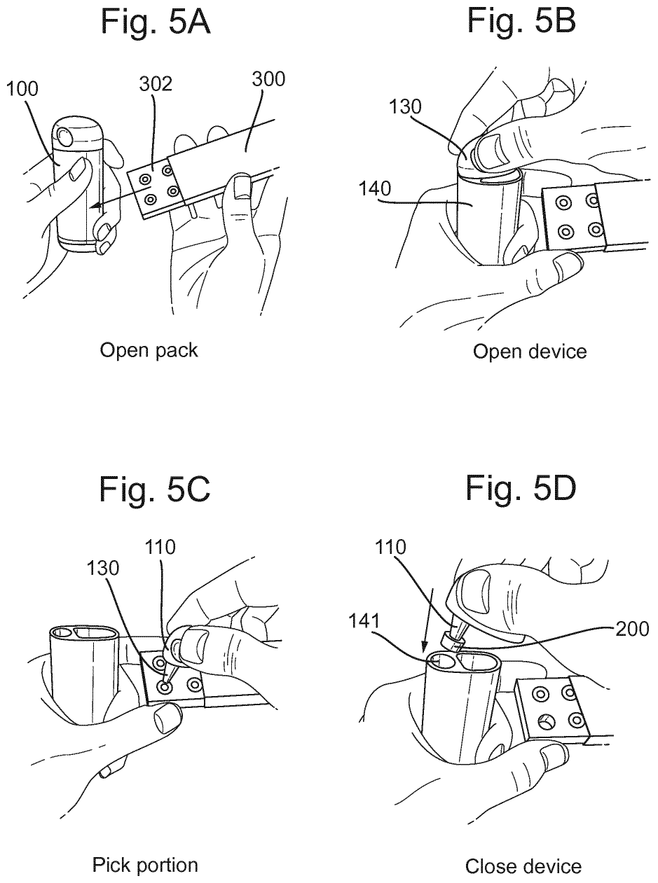

FIG. 5 illustrates the full process for using the aerosol generating system according to the present invention. This shows how exchange of the consumable 200 may be carried out in a straightforward manner, which requires little dexterity and can be achieved without any skin contact with the consumables 200. In particular, in FIG. 5A the tray 302 is slid out of the external packaging of the consumable package 300, the heating chamber lid 130 is then removed from the body 140 of the device 100 by rotating and lifting it away, as previously described. FIG. 5C shows how the lid may be easily manoeuvred by holding the lid between the thumb and forefinger using the indentations 132 and the protrusion 110 may be engaged with the upward facing opening 201 of the consumable 200 within the consumable array of the packaging 300. The consumable 200 is retained on the protrusion 110 as shown in FIG. 5D. The lid 130 is then returned to the device 100 by placing the protrusion 110 and the retained consumable 200 into the opening 141 of the heating chamber 120, as shown in FIG. 5D. The aerosol generating device 100 is then turned on to activate the heaters 121 of the heating chamber 120. In the example of FIG. 5E, this is achieved by pressing downwards on the lid 130 in a direction corresponding to the elongate axis of the body 140 of the device 100. The device is then turned around as shown in FIG. 5F such that the user may inhale the generated vapour from the mouthpiece 150. When the consumable is expended, which may be indicated by a light or other indication means of the device 100, the heating chamber 120 is opened as shown in FIG. 5G by removing the lid 130 and the spent consumable 200 may be simply removed by tapping the lid 130 to dislodge the dispensed consumable 200 from the protrusion 110. In other examples of the invention the device 100 may further comprise a release means, for example a button which causes the spent consumable to be released from the protrusion 110. In the example of FIGS. 1-5, this can take the form of a mechanism which retracts the protrusion 110 fully or partially in order to displace the spent consumable 200. After use, the device is closed by returning the protrusion into the heating chamber, thus closing the lid 130.

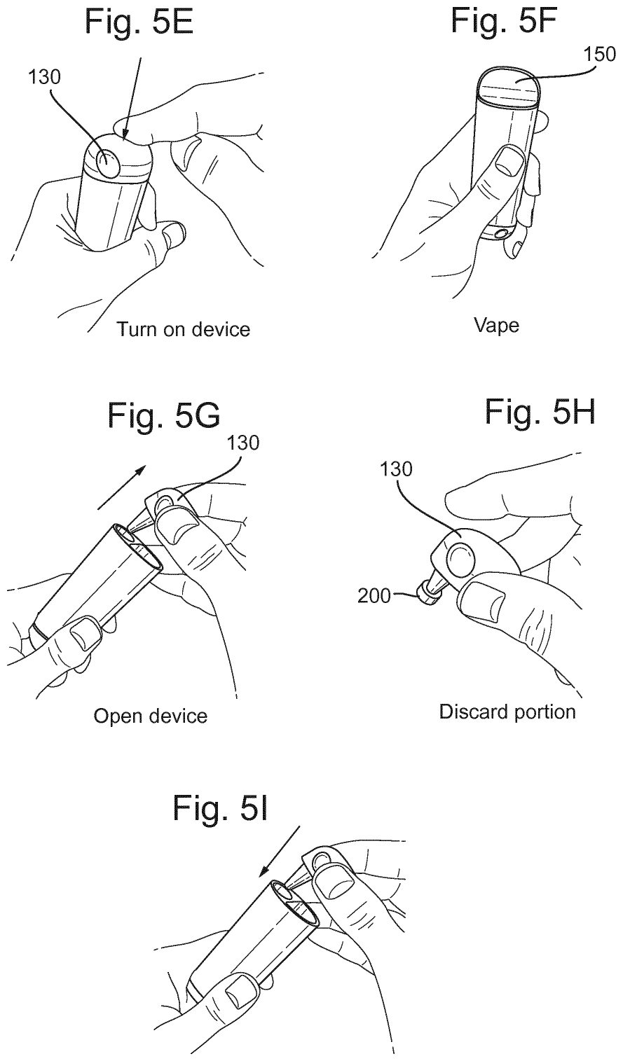

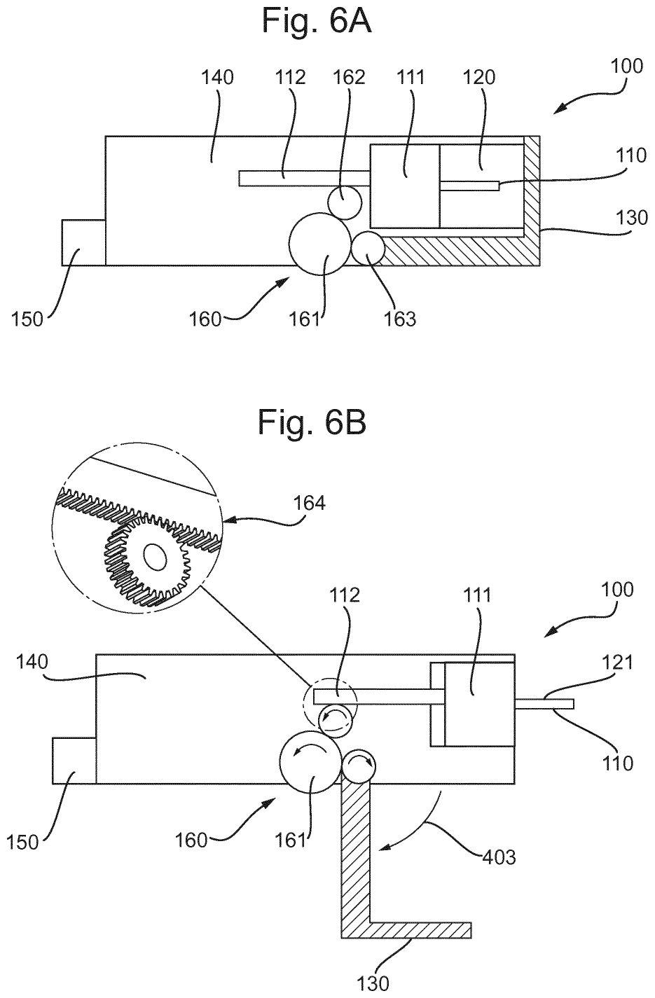

FIG. 6 shows a schematic illustration of an alternative example of the aerosol generating device 100 according to the present invention. In this example, the consumable picker is also provided by a protrusion 110 but, instead of being attached to the lid 130, the protrusion is provided on a piston 111 having a piston shaft 112. Movement of the consumable picker 110 between the first external position (shown in FIG. 6B) and the second internal position (shown in FIG. 6A) is provided by means of a system of gears 160 which are coupled to the piston shaft 112 to drive the piston 111 in a direction aligned with the elongate access of the body 140 of the device 100. The lid 130 forms a separate component to the consumable picker 110 but, in this example, the lid 130 is also coupled to the system of gears 160 such that movement of the lid 130 into the open position, shown by arrow 403, simultaneously moves the piston from the retracted position of FIG. 6A into the extended position of FIG. 6B.

The system of gears 160 may include an actuator, in this case actuating wheel 161, which extends partially to the outside of the body 140 as shown in FIG. 6. The user may then rotate this wheel 161 which simultaneously rotates a piston shaft gear 162 and a lid gear 163 which engage respectively with the piston shaft 112 and a portion of the lid 130. This may be by means of intercalating ridges as shown in the expanded image of the gears 164. Therefore, when the device is in the closed position of FIG. 6A and the consumable picker 110 is retracted within the heating chamber 120, the user may move the actuating wheel 161 which drives the movement of the piston 111 in a direction corresponding to the elongate access of the device such that the protrusion 110 extends out of the heating chamber as shown in FIG. 6B. Simultaneously as the piston moves the lid 130 opens from the closed position to an open position as shown in FIG. 6B. In this configuration the user may move the picker to first external position by opening the lid instead of rotating the wheel 161.

Once in the open position in which the protrusion 110 extends out of the device 100, the device 100 as a whole may be used to move towards and select a consumable 200, for example from the consumable package 300 shown in FIG. 4. This has the advantage that rather than the process of FIG. 5 in which the device body 140 and lid 130 must be held separately, in this arrangement since the lid 130 is attached to the body 140 at all times and the protrusion is provided on the device itself, the device can simply be held in one hand and orientated appropriately to engage a consumable 200 before the protrusion 110 is retracted back into the heating chamber 120, and the lid closed for heating.

The example of FIG. 6 has a similar arrangement of battery connected to the heater. The heater 121 may be arranged within the body 140 of the device 100 around the heating chamber 120 in a similar manner to FIG. 1E. However, in this example the protrusion 110 itself is a heating element 121. This is possible because the protrusion forms part of and is connected to the main body 140 of the device such that it is straight forward to provide connections to the battery 160 containing the body 140. This arrangement has the advantage that once the protrusion 110 is within the consumable 200 and the piston is retracted back into the chamber 120, with the lid 130 closed, the consumable 200 is heated from the inside thus providing uniform heating of the material. Furthermore, heating the consumable from within is more efficient as less heat escapes and accordingly the heating chamber does not need to be as well insulated.

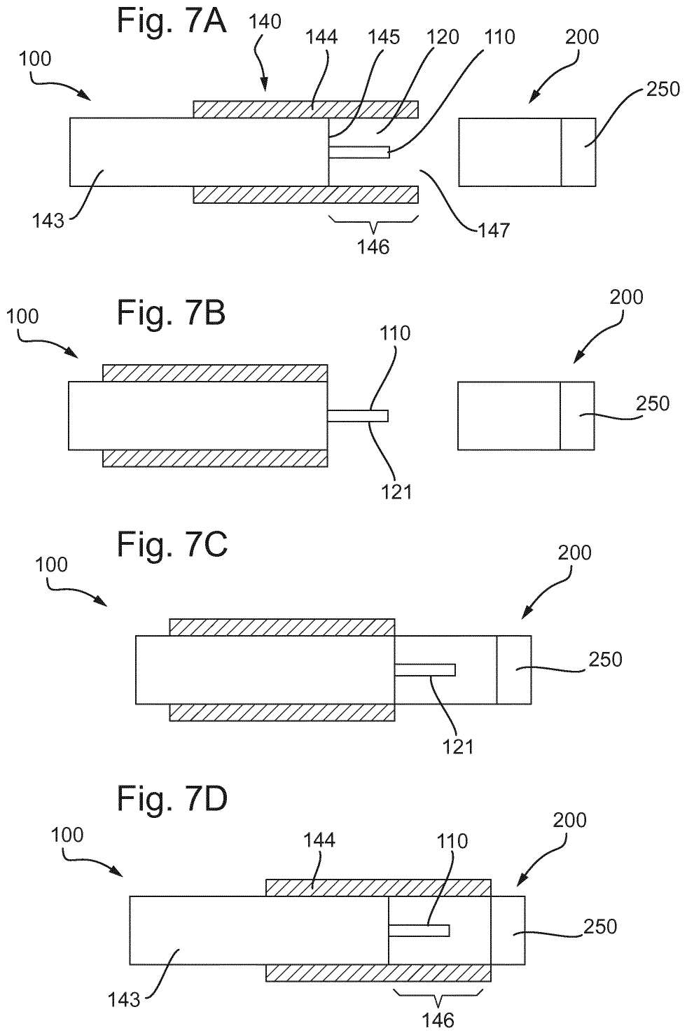

FIG. 7 shows another alternative example of the aerosol generating device according to the present invention. In this example, the body 140 of the device 100 is formed of a core component 143 and a sleeve 144, wherein the body is moveable in an axial direction within the sleeve. In particular, the core component of the body 143 may be provided a substantially cylindrical body portion which is moveable within the sleeve 144 in the direction corresponding to the elongate access of the device 100. The protrusion 110 is provided on an axial end face 145 of the core component 143, such that it is substantially aligned with the elongate access of the body 140. Movement of the consumable picker 110 between the first external position, shown in FIG. 7B, and the second, internal position, shown in FIG. 7A, is provided by this sliding movement of the core 143 relative to the sleeve 144. In particular, when the core is moved such that the consumable picker is retracted within an extending portion 146 of the sleeve the heating chamber is defined by the end face 145 of the core component 143 and the extending portion of the surrounding sleeve 146.

In this way, by moving the core 143 relative to the sleeve 144 such that the protrusion 110 is extended outside of the chamber 120, a consumable 200 may be engaged with the protrusion before the core is retracted back within the sleeve causing the consumable 200 retained on the protrusion 110 to be moved into the heating chamber 120 for heating, as shown in FIG. 7D. Again in this example the protrusion 110 is also a heating element 121 such that heating of the consumable 200 takes places from within the consumable 200. However, equally, the heater 121 may be provided by heating elements placed on the inside walls of the portion of the sleeve 146. In this example, the device 100 does not include a mouthpiece 150 itself, but a mouthpiece 250 is provided on the consumable 200. In particular the consumable comprises a cylindrical body of aerosol generating material arranged to fit within the cylindrical space of the heating chamber 120 defined by the extending sleeve potion 146 and the end face 145 of the core. The consumable 200 further comprises a filter portion 250 at one axial end of the consumable which provides a mouthpiece 250.

As can be seen from FIG. 7D when the consumable 200 is engaged with the protrusion 110 and it is retracted back within the heating chamber 120 formed by the portion 146 of the sleeve 144 the consumable mouthpiece 250 remains extended out of the opening 147 of the sleeve 144. Once the heating element 121 is activated, such that an aerosol is generated from the material of the consumable 200, the aerosol may be inhaled via the mouthpiece 250 of the consumable. Equally, the opening to the sleeve 147 may be provided with a lid and the mouthpiece may be provided at the other end of the device, as with the previously described examples of the invention.

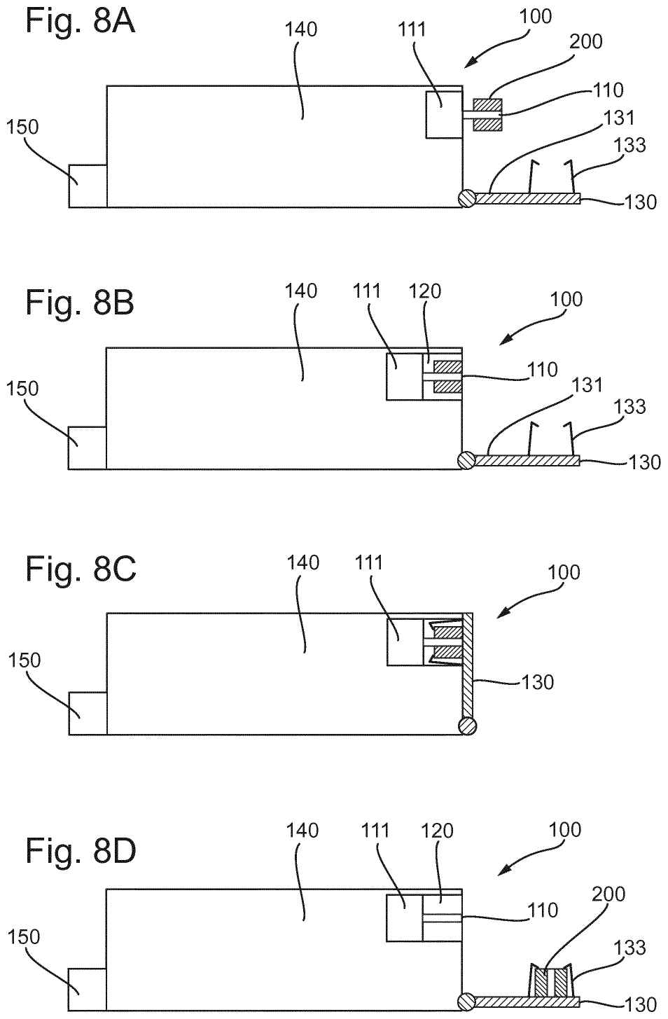

FIG. 8 shows a further example of the aerosol generating device 100 according to the present invention. This example has a similar arrangement to that of FIG. 6 and the description of the features of FIG. 6 above applies equally to FIG. 8. In particular, the device 100 has a piston 111 with a consumable picker 110 in the form of a protrusion provided on one side of the piston 111. The piston is moveable within the body 140 of the device 100 such that the protrusion 110 moves between an internal position as shown in FIG. 8b in which the protrusion is retracted such that it lies within the heating chamber 120 and an external position, as shown in FIG. 8a, in which the protrusion is external to the device such that it may be used to engage a consumable. As with FIG. 6, the device includes a heating chamber lid 130 which is moveable between an open position as shown in FIG. 8a and a closed position as shown in FIG. 8c.

The device of FIG. 8 differs from that of FIG. 6 in that it further comprises an engagement element 133 arranged to remove the retained consumable 200 from the consumable picker 110. In the example of FIG. 8 the engagement element 133 is in the form of a grip 133 disposed on an inner surface 131 of the lid 130. The grip 133 is configured to grip the outer surfaces of the consumable such that it may be removed from the protrusion 110.

As shown in FIG. 8a the device 100 may be operated by firstly moving the piston in the direction corresponding to the elongate axis of the body 140 of the device 100 such that the protrusion 110 is moved to the first external position. In this arrangement the external protrusion 110 may be used to engage a consumable 200 as shown in FIG. 8a.

The piston 111 may then be retracted in a direction corresponding to the elongate axis of the device 100, moving the protrusion 110 and the retained consumable 200 into the heating chamber 120, as shown in FIG. 8b.

When the retained consumable 110 is withdrawn within the heating chamber the lid 130 may be closed, as shown in FIG. 8c, to enclose the retained consumable 200 within the heating chamber 120. The heater may then be activated when the consumable is retained as shown in FIG. 8c in order to generate the inhalable vapour by heating the consumable. As described above in reference to FIG. 6 the protrusion itself may provide the heating element such that the consumable 200 is heated from within thereby providing advantages in terms of the uniformity of the heating and the efficiency as less heat is lost through the body 140 of the device 100. As the lid 130 is moved to the closed position the gripping element 133 engages the consumable by gripping the external surfaces of the consumable while it lies within the heating chamber 120.

After the consumable is depleted, the lid 130 may be moved to the open position as shown in FIG. 8d. As the lid is opened the gripping force applied by the gripping element 133 extracts the spent consumable 200 from the heating chamber 120, thereby releasing the consumable from its position on the protrusion 110. The consumable 200 may then be removed from the gripping element 133 by shaking the device 100, tapping it to dislodge the consumable 200 or via an automated means to release the gripping element thereby releasing the spent consumable 200.

As described with reference to FIG. 6, a user actuation means may be provided to control both the opening and closing of the lid and the movement of the piston 111, either simultaneously or independently as shown in FIG. 8.

With the aerosol generating device and aerosol generating system of the present invention, an improved consumable exchange process is provided. In particular, a consumable may be engaged with the consumable picker and moved into the heating chamber, where it remains engaged with the consumable picker. It can thus be removed in a straightforward manner by removing the consumable picker and dislodging the spent consumable. The entire consumable exchange process can thus be performed without requiring the user to touch the consumable and significantly reducing the complexity of the process and the dexterity required to carry it out.

1. An aerosol generating device comprising:
a heating chamber arranged to receive a consumable and heat the consumable to generate an inhalable vapour;
a consumable picker arranged to engage and retain a consumable such that it may pick up a consumable, wherein the consumable picker is movable relative to the heating chamber between:
a first, external position in which the consumable picker is positioned external to the device; and
a second, internal position in which the consumable picker lies within the heating chamber; such that
a user may engage a consumable with the consumable picker in the first position to pick up the consumable with the consumable picker and subsequently move the consumable picker to the second position, thereby moving the consumable picker with the retained consumable into the heating chamber for heating.

2. The aerosol generating device of claim 1, wherein the consumable picker comprises a protrusion.

3. The aerosol generating device of claim 2, wherein the protrusion has a tapered shape.

4. The aerosol generating device of claim 1, further comprising:
a heating chamber lid, moveable between an open position to allow access to the heating chamber and closed position in which the heating chamber is enclosed; wherein
the consumable picker is coupled to the heating chamber lid such that movement of the lid between the open and closed position moves the consumable picker between the external and internal position to simultaneously insert the consumable picker into the chamber and close the heating chamber opening.

5. The aerosol generating device of claim 4, wherein the consumable picker forms part of the heating chamber lid and extends from an internal surface of the heating chamber lid such that when the heating chamber lid is in the closed position the consumable picker extends into the heating chamber.

6. The aerosol generating device of claim 4, wherein the consumable picker is a separate component to the lid and the consumable picker and the lid are mechanically coupled such that movement of the consumable picker between the external and internal position drives movement of the lid between the open and closed position and/or movement of the lid between the open and closed position drives movement of the consumable picker between the external and internal position.

7. The aerosol generating device of claim 6, comprising a user actuation means arranged to drive the movement of the heating chamber lid and the consumable picker.

8. The aerosol generating device of claim 1, wherein the device further comprises:
an elongate body and a sleeve, the elongate body moveable within the sleeve in a direction corresponding to the elongate axis; wherein
the consumable picker forms part of the body with the protrusion extending from one end of the body; such that
relative movement of the sleeve and body provides movement between the first position in which the protrusion extends out of the sleeve and the second position in which the protrusion is retracted within the sleeve.

9. The aerosol generating device of claim 8, wherein the heating chamber is provided by the internal volume defined by an end face of the body and the surrounding internal surface of the sleeve when the consumable picker is in the second position in which the protrusion is retracted within the sleeve.

10. The aerosol generating device of claim 1, wherein the consumable picker comprises a heating element arranged to heat the consumable when received in the heating chamber.

11. The aerosol generating device of claim 1, wherein the device further comprises a releasing means configured to release the consumable from consumable picker upon actuation by the user.

12. An aerosol generating system comprising:
the aerosol generating device according to claim 1;
a consumable formed such that it will be retained after being engaged by the consumable picker.

13. The aerosol generating system according to claim 12, wherein the consumable picker comprises a protrusion and the consumable has a central cavity shaped such that the protrusion of the consumable picker may be inserted into the cavity and the consumable retained on the protrusion.

14. The aerosol generating system according to claim 12, further comprising:
a consumable package arranged to hold a plurality of consumables, each in a corresponding orientation such that a face of the consumable which is capable of being engaged with the consumable picker faces outwards, allowing a user to select a consumable by engaging the face with the consumable picker.

15. The aerosol generating system according to claim 12, wherein the consumable comprises a mouthpiece such that, when the consumable is engaged by the consumable picker and moved to the internal position within the chamber, the mouthpiece extends outside of the chamber allowing a user to inhale the generated vapour through the consumable.
